(19) United States
(12) Patent Application Publication
(10) Pub. No.: US 2025/0170050 A1
(43) Pub. Date: May 29, 2025

(54) Use of Nordic plant-based ingredients for supporting a healthy skin microbiome

(71) Applicant: Lumene Oy, Espoo (FI)

(72) Inventors: Saija Haapa-Paananen, Espoo (FI); Tiina Isohanni, Espoo (FI); Päivi Mäkinen, Espoo (FI); Ingela Palmujoki, Espoo (FI); Terhi Pesonen, Espoo (FI); Elina Sahramo, Espoo (FI); Kristofer Vänttinen, Espoo (FI)

(21) Appl. No.: 18/835,372

(22) PCT Filed: Feb. 2, 2023
(86) PCT No.: PCT/FI2023/050064

§ 371 Date: Aug. 2, 2024

(30) Foreign Application Priority Data
Feb. 2, 2022 (FI) 20225087

(51) Int. Cl. A61K 8/9789; A61K 8/60; A61K 8/9728; A61Q 19/00

(52) U.S. Cl. CPC A61K 8/9789; A61K 8/60; A61K 8/9728; A61Q 19/00; A61K 2800/80

(57) ABSTRACT

According to an example aspect of the present invention, there is provided non-therapeutic use of an effective amount of substances derived from Nordic plants, such as birch sap, fermented oat extract, extracts or dried powders derived from meadowsweet, roseroot, and/or chaga and any combinations thereof, optionally in combination with oat xylitol, as cosmetic agents supporting a healthy skin microbiome, and a corresponding non-therapeutic method. The non-therapeutic method of the invention preferably comprises increasing the ratio of S. epidermis to C. acnes in human skin microbiome by applying on a human skin an effective amount of cosmetic composition comprising any one of the above-mentioned ingredients or their combinations.

### FIELD

The present invention relates to the field of cosmetic industry. In particular, the present invention relates to cosmetic use of an effective amount of substances derived from Nordic plants as agents supporting a healthy skin microbiome and thus improving skin appearance. The invention also relates to a non-therapeutic method for increasing the ratio of commensal bacteria to harmful or pathogenic bacteria in human skin microbiome, in particular for increasing the ratio of Staphylococcus epidermidis to Cutibacterium acnes (Propionibacterium acnes). The method comprises a step of applying on the skin a topical cosmetic composition comprising an effective amount of substance(s) derived from Nordic plants, wherein said substances comprise extracts or dried extracts of meadowsweet and roseroot, optionally with chaga, fermented oat or its dried form, birch sap or its dried form, or any combinations thereof, and further optionally in combination with oat xylitol.

### BACKGROUND

Microbiome is generally defined as an “ecological community of commensal, symbiotic and pathogenic microorganisms influenced by environmental variables”. Human skin is colonized by millions of bacteria, fungi and viruses that compose the skin microbiome. As human skin functions as the exterior interface of the human body with the environment, it acts as a physical barrier to prevent the invasion of foreign pathogens while providing a home to the commensal microbes.

A healthy human skin shows a very diverse and heterogeneous microbiome while in case of skin problems the balance between commensal microbes and pathogens may be disturbed. The skin microbiome is typically populated with a wide variety of micro-organisms and there are variations in the relative abundance of bacterial taxa populating dry, moist or sebaceous areas on the skin (Byrd et al. 2018). Dry skin areas, e.g. forearms and legs have the most diverse microbiota. These areas are dominated by Propionibacterium, Corynebacterium, Staphylococcus, Streptococcus, Acinetobacter, and many other bacterial species in lower amounts (San Miguel and Grice 2015). Corynebacterium spp. are common in moist areas of the skin, e.g. axillary area (Callewaert et al. 2013).

The skin microbiome continues to evolve throughout life. It is affected by skin's condition, including the amount of water, pH, lipids, proteins, humectants, etc. The human skin microbiome is also affected by external environment and people we get in contact with, since we may also adopt bacteria from others. Moreover, the skin microbiome is also affected by the skin care products we use. Exposure to preservatives from cosmetics may kill microbes. Certain pH ranges of cosmetic products may favour growth for specific bacteria and reduce the growth of others.

Commensal bacteria are bacteria inhabiting the host for long-periods without causing harm or benefit, as opposed to harmful bacteria that may cause diseases. Prebiotics are typically carbohydrates that stimulate the growth of commensal bacteria, while probiotics generally include “good” bacteria that may prevent or reduce the growth of harmful bacteria.

Staphylococcus epidermidis (S. epidermidis) is one of the most common skin commensal bacteria. Colonisation of S. epidermidis is beneficial for the skin as several studies have shown that it can act as probiotic bacteria preventing the growth of harmful or pathogenic bacteria on the skin. S. epidermidis has been shown to inhibit biofilm formation of the pathogenic Staphylococcus aureus bacteria by secreting a serine protease (Iwase et al., 2010). In addition, Wang et al (2014) showed that S. epidermidis is able to ferment glycerol present naturally in skin to restrict the growth of Propionibacterium acnes (now known as (′utibacterium acnes), skin bacteria which is known to cause acne vulgaris in skin. (′utibacterium acnes is a normally harmless bacterium on the skin known to be associated with acne. This bacterium thrives on adolescent skin because of its increased fat content, which favours this bacterium. Acne (Acne vulgaris) is a common skin condition caused by excessive sebum secretion by the sebaceous glands or thickening of the stratum corneum of the top layer of the skin. In this case, the sebum cannot be excreted freely from the sebaceous gland. Sebum is an important nutritional factor for Cutibacterium acnes. When sebum is abundant on the skin, there are also many viable (′. acnes bacteria.

It would thus be beneficial to find means to support the growth of commensal bacteria, such as S. epidermidis, and hence to improve skin appearance and limit the growth of harmful bacteria in human skin microbiome. In terms of cosmetic skin care, it would be particularly beneficial to achieve a good balance between S. epidermidis and C. acnes in human skin. A balanced skin microbiome is more capable to resist signs of aging, while also reducing dryness, redness and irritation of the skin. Therefore, prebiotics that would increase the amounts of S. epidermidis in the skin and reduce C. acnes bacteria are needed in cosmetic, non-therapeutic skin care.

A large number of cosmetic compositions, which contain plant-based ingredients are on the market. A moisturizing cosmetic composition comprising birch sap, possibly in combination with moisturizing xylitol, is disclosed in US 2020/004663 A1. Various berry-derived substances for cosmetic use are disclosed for example in EP 3365068, EP1337224, and EP2814242. FR 2897778 A1 discloses a process for obtaining an active ingredient from meadowsweet powder for improving the condition of oily and/or acne-prone skin. The use of fermented oat extract and oat xylitol in cosmetics is also known or suggested.

However, the non-therapeutic use of Nordic plant-based ingredients selected from those disclosed in the present application in cosmetic compositions to support a healthy skin microbiome by increasing the ratio of commensal bacteria to harmful or pathogenic bacteria in human skin microbiome or the corresponding non-therapeutic method have not been disclosed or suggested in prior art.

### SUMMARY OF THE INVENTION

The invention is defined by the features of the independent claims. Some specific embodiments are defined in the dependent claims.

The present invention is based on the finding that substances derived from Nordic plants, in particular extracts from meadowsweet and roseroot or their dried powder forms, as well as birch sap, fermented oat extract and chaga or their dried powder forms, and any combinations thereof, optionally in combination with oat xylitol, promote the growth of commensal bacteria in relation to harmful or pathogenic bacteria of the human skin. In particular, the above-mentioned plant-based ingredients are derived from plants growing in the Nordic area.

According to a first aspect of the present invention, there is thus provided the non-therapeutic use of substances derived from Nordic plants in cosmetic compositions for increasing the ratio of commensal bacteria to harmful or pathogenic bacteria in human skin microbiome, wherein the substances derived from Nordic plants comprise extracts or dried extracts of meadowsweet and roseroot and any combinations thereof, optionally in combination with oat xylitol.

According to a second aspect of the present invention, there is provided a non-therapeutic cosmetic method for increasing the ratio of commensal bacteria to harmful or pathogenic bacteria in human skin microbiome, the method comprising a step of applying on the skin a topical cosmetic composition comprising an effective amount of substance(s) derived from Nordic plants, wherein said substances comprise extracts or dried extract powders of meadowsweet and roseroot and any combinations thereof, optionally in combination with oat xylitol.

According to another aspect of the present invention, there is provided a cosmetic composition for use in a non-therapeutic method for increasing the ratio of commensal bacteria to harmful bacteria in human skin microbiome, wherein the cosmetic composition comprises an effective amount of at least one substance derived from Nordic plants, wherein said substance(s) comprise extracts or dried extracts of meadowsweet and roseroot and chaga, fermented oat and its dried form, birch sap and its dried form, and any combinations thereof, optionally in combination with oat xylitol.

Embodiments of the invention comprise for example the above mentioned non-therapeutic method, a cosmetic composition and use for increasing the ratio of Staphylococcus epidermidis to harmful or pathogenic bacteria in human skin microbiome, in particular for increasing the ratio of Staphylococcus epidermidis to Cutibacterium acnes (Propionibacterium acnes).

In a further aspect, the invention relates to the non-therapeutic use of meadowsweet extract and roseroot extract and/or dried forms thereof, optionally in combination with oat xylitol, in combination with further substances derived from Nordic plants, wherein said substances are selected from chaga, fermented oat and its dried form, birch sap and its dried form and any combinations thereof in a cosmetic composition for increasing the ratio of commensal bacteria to harmful or pathogenic bacteria in human skin microbiome.

In a still further aspect, the invention relates to the non-therapeutic use of birch sap, optionally in combination with oat xylitol, in a cosmetic composition for increasing the ratio of commensal bacteria to harmful or pathogenic bacteria in human skin microbiome.

Considerable advantages are obtained by the invention. First, when the growth of harmful or pathogenic bacteria is limited and the skin microbiome is balanced, the skin is able to effectively act as a physical barrier to prevent the skin and the body from the undesired cosmetic effects of harmful bacteria.

Second, a healthy and balanced skin microbiome helps to keep the surface of skin smooth and strong, while also reducing dryness, redness and irritation of the skin. Further, a strong and balanced skin microbiome is more capable to resist signs of aging.

Third, various cosmetic skin conditions like mild acne may be alleviated with cosmetics by increasing the ratio of commensal bacteria in human skin. For example, atopic skin is typically rich in S. aureus. Balancing the skin microbiome by increasing the ratio of S. epidermidis to S. aureus may thus alleviate the symptoms of mildly atopic skin. Mild forms of atopic, dry skin as well as skin prone to acne can benefit from cosmetic products balancing the skin microbiome and thus improving skin appearance.

Moreover, a balanced skin microbiome provides protection from external aggressors, such as air pollution. Air pollutants may cause the complexion to appear uneven and dull and the skin feels uncomfortable.

20 In some embodiments, the prebiotic effect of substances derived from Nordic plants may also alleviate undesirable skin odours caused by unbalanced skin microbiome, typically in the underarm area.

Further features and advantages of the present technology will appear from the following description of some embodiments.

### BRIEF DESCRIPTION OF THE DRAWINGS

FIG. 1 illustrates the effects of xylitol (0.5%) and birch sap (20%, 10%. 1% concentration) on S. epidermidis (FIGS. 1A) and C. acnes (FIG. 1B) growth in vitro.

FIG. 2 illustrates the effects of xylitol (0.5%) and birch sap (20% concentration) on S. epidermidis (FIGS. 2A) and C. acnes (FIG. 2B) growth in vitro.

FIG. 3 illustrates growth of S. epidermidis (FIGS. 3A) and C. acnes (FIG. 3B) on 0.5% Xylitol+birch sap (20%/10%/1%)+0.5% fermented oat extract (Ferment N) combinations. The C. acnes data is from two different 96-well plates.

FIG. 4A illustrates the effect of different concentrations of meadowsweet alone on S. epidermidis growth under laboratory conditions (FIG. 4A). Effect of different concentrations of meadowsweet and xylitol (0.5%) on S. epidermidis growth under laboratory conditions is shown in FIG. 4B.

FIG. 5 illustrates the effect of different concentrations of meadowsweet alone on C. acnes bacterial growth under laboratory conditions (FIG. 5A). Effect of different concentrations meadowsweet and xylitol (0.5%) on C. acnes bacterial growth under laboratory conditions is shown in FIG. 5B.

FIG. 6 illustrates the effect of arctic roseroot on the growth of S. epidermidis and C. acnes bacteria alone (FIG. 6A) and in combination with 0.5% xylitol (FIG. 6B).

FIG. 7 further illustrates the effect of arctic roseroot (0.5%) on the growth of S. epidermidis (FIGS. 7A) and C. acnes (FIG. 7B) bacteria. The results of S. epidermidis are from two different plate series, therefore the control and glucose 1% control are presented twice.

FIG. 8 illustrates the effect of different concentrations of chaga alone (FIG. 8A) or in combination with 0.5% xylitol (FIG. 8B) on S. epidermidis bacterial growth under laboratory conditions.

FIG. 9 illustrates the effect of different concentrations of chaga alone (FIG. 9A) or in combination with 0.5% xylitol (FIG. 9B) on C. acnes bacterial growth under laboratory conditions.

FIG. 10A illustrates the results of quantification of total flora with qPCR from swaps taken at time point 0 (TO) and after 1 month (T28) of use of a cosmetic serum containing 0.5% meadowsweet and 0.5% roseroot. FIG. 10B shows quantification of S. epidermidis and C. acnes with qPCR and calculated in relation to total flora, wherein evolution % as (T28-TO)/TO was calculated individually for each subject. In FIG. 10C a mean of each bacteria was calculated. T-test gave a statistically significant difference between the C. acnes and S. epidermidis mean evolution % values (70% difference in the means).

### EMBODIMENTS

### Definitions

In the present context, the term “Nordic plants” or “Nordic wild plants” refers mainly to plants naturally growing in the Nordic area without agriculture or gardening. However, to ensure diversity of Nordic nature, sources of natural plant extracts and other components may also be cultured. The natural ingredients may thus originate from farming methods or from wild harvesting in compliance with national legislation.

The Nordic area refers to a geographical region in Northern Europe and the North Atlantic. Preferably, the “Nordic plants” referred herein include plants growing in the Nordic countries, which by definition include Denmark, Finland, Iceland, Norway, and Sweden, as well as Greenland, the Faroe Islands, the Åland islands and Svalbard archipelago.

In the present context, “commensal bacteria” refer to bacteria inhabiting the human skin microbiome for long periods without causing harm or benefit, as opposed to harmful or pathogenic bacteria that sometimes may cause disease. One of the most common commensal skin bacteria is Staphylococcus epidermidis. Also e.g. Staphylococcus hominis has proven beneficial to human skin (Byrd, 2018). However, it is also known that in certain contexts, bacteria that are ordinarily beneficial to their hosts may become harmful or pathogenic.

In the present context, “harmful or pathogenic bacteria” include such bacteria that are potential to cause harmful cosmetic symptoms, in particular when their population or share in the skin microbiome increases. Examples of typical harmful or pathogenic bacteria of human skin microbiome include but are not limited to Cutibacterium acnes (Propionibacterium acnes), Staphylococcus aureus, Pseudomonas aeruginosa, Escherichia coli, Corynebacterium mastitidis, and Corynebacterium bovis, in particular Cutibacterium acnes (Propionibacterium acnes) and Staphylococcus aureus.

An “effective amount” refers to an amount adequate to accomplish a desired or intended result.

Within the present context, “oat xylitol” or “oat derived xylitol” means xylitol obtained by processing Nordic oat hulls, in particular Nordic oat hulls obtained as a side stream from oat milling process. In brief, oat hulls are hydrolysed, the obtained hydrolysate is separated to a solid fraction and to a carbohydrate containing liquid fraction, which is rich in xylose and may be used in the production of xylitol.

It has been found that substances derived from Nordic plants, in particular extracts from meadowsweet and roseroot, chaga, birch sap, fermented oat extract or their dried forms and any combinations thereof, optionally in combination with oat xylitol, promote the growth of commensal bacteria in relation to harmful or pathogenic bacteria of the human skin.

Therefore, in one embodiment the present invention provides the non-therapeutic use of the above-mentioned ingredients in cosmetic compositions for increasing the proportion of Staphylococcus epidermidis in relation to harmful or pathogenic bacteria of human skin.

In a normal situation, S. epidermidis is a major inhabitant of the skin, and in some areas it may make up more than 90 percent of the resident aerobic flora. Another species of Staphylococcus sp, namely Staphylococcus aureus, is extremely common (80 to 100 percent) on the skin of patients with certain dermatologic diseases, such as atopic dermatitis. Thus colonization by S. aureus on the skin is one of the factors that can worsen general condition of the skin, including common drying of the skin.

Anaerobic diphtheroids (coryneforms) are most common in areas rich in sebaceous glands. Although the name Corynebacterium acnes was originally used to describe skin anaerobic diphtheroids, these are now classified as Propionibacterium acnes and as P. gramilosum. P. acnes is seen more frequently than P. gramilosum in acne lesions and is probably involved in acne pathogenesis (Davis, 1996). Acne is presented in many different grades from mild to more severe forms, and the milder forms can benefit from cosmetic products with suitable ingredients.

In vitro studies have shown that the above-mentioned cosmetic ingredients derived from Nordic plants increase the proportion of commensal bacteria, in particular S. epidermidis, in relation to harmful or pathogenic bacteria, typically (′. acnes. This finding finds use in non-therapeutic cosmetic applications, such as in cosmetic compositions and in a cosmetic method for increasing the ratio of commensal bacteria to harmful or pathogenic bacteria in human skin microbiome.

Thus in one embodiment, the invention relates to non-therapeutic use of cosmetic compositions comprising substances derived from Nordic plants, wherein said substances comprise extracts or dried extracts of meadowsweet and roseroot, optionally with chaga, fermented oat or its dried form, birch sap or its dried form, or any combinations thereof, and further optionally in combination with oat xylitol, and a cosmetically acceptable carrier, for increasing the ratio of Staphylococcus epidermidis to harmful or pathogenic bacteria.

In particular, the above-mentioned cosmetic compositions and substances find use in increasing the ratio of Staphylococcus epidermidis to Cutibacterium acnes (Propionibacterium acnes).

Therefore, in one embodiment the invention comprises a non-therapeutic cosmetic method for increasing the ratio of commensal bacteria to harmful or pathogenic bacteria in human skin microbiome, the method comprising applying on the skin a topical cosmetic composition comprising an effective amount of substance(s) derived from Nordic plants, wherein said substances comprise extracts or dried extracts of meadowsweet and roseroot, optionally in combination with oat xylitol.

In a further embodiment, the invention comprises a cosmetic composition for use in a non-therapeutic method for increasing the ratio of commensal bacteria to harmful bacteria in human skin microbiome, wherein the cosmetic composition comprises an effective amount of at least one substance derived from Nordic plants, wherein said substance(s) comprise extracts or dried extracts of meadowsweet and roseroot, optionally in combination with oat xylitol.

In one or more embodiments, the cosmetic composition may comprise further substances derived from Nordic plants, wherein said substances are selected from chaga, fermented oat and its dried form, birch sap and its dried form, and any combinations thereof.

Typically, the effective amount of the above-mentioned substance(s) derived from Nordic plants is 0.01-70% by weight of the cosmetic composition, preferably 0.1-20% by weight of the cosmetic composition.

In vitro studies have shown that the above-mentioned substances from Nordic plants may increase the ratio of S. epidermidis to C. acnes from 1:1 to about 20:1 within 48 hours in vitro, preferably from about 1:1 to at least 10:1 within 24 hours in vitro. During one-month non-therapeutic use of cosmetic compositions comprising the above-mentioned substances derived from Nordic plants the ratio of S. epidermidis to C. acnes may increase for example from 1:1 to about 5:1 when the cosmetic composition is applied twice a day on the human skin.

Arctic meadowsweet (Filipendula ulmaria, old name Spiraea ulmaria)

Meadowsweet or mead wort is a herbaceous plant in the family Rosaceae that grows typically in damp meadows. Meadowsweet flowers from early summer to early autumn with creamy-white flowers, having a very strong, sweet smell. Meadowsweet is known to contain salicylic acid, which is a well-known disinfectant and anti-bacterial agent.

In one or more embodiments, the cosmetic composition for use in the method of the invention comprises meadowsweet extract or dried meadowsweet extract (meadowsweet powder), preferably in an amount of 0.01-10% by weight of the composition, more preferably in an amount of 0.1-5% by weight of the composition, and a cosmetically acceptable carrier.

The meadowsweet extract used in the present invention is typically obtained from flowers of meadowsweet by a method comprising the steps of extracting the flowers with a solvent, preferably with circulating glycerin: water, with water: butylene, or with pentyleneglycol, propanediol or ethanol, more preferably with circulating glycerine: water at room temperature, and optionally drying the obtained meadowsweet extract, typically by spray-drying, to obtain meadowsweet powder.

Artic roseroot (Rhodiola rosea)

Arctic roseroot is a perennial flowering plant in the family Crassulaceae. It grows naturally in the Arctic regions of Nordic countries. Although there appears to be no scientific clinical evidence of its effectiveness, roseroot has been widely used in traditional medicine.

In one or more embodiments, the cosmetic composition for use in the method of the invention comprises roseroot extract or dried roseroot extract, preferably in an amount of 0.01-10% by weight of the composition, more preferably in an amount of 0.1-5% by weight of the composition, and a cosmetically acceptable carrier.

The roseroot extract used in the present invention is typically obtained from roots of roseroot plants by a method comprising the steps of extracting the roots with a solvent, preferably with circulating glycerine: water, with water: butylene, with pentyleneglycol, propanediol or ethanol, more preferably with circulating glyecerine: water at room temperature, and optionally drying the obtained roseroot extract, typically by spray-drying, to obtain roseroot powder.

According to one aspect, the invention relates to a combined non-therapeutic use of meadowsweet extract and roseroot extract and/or dried forms thereof, optionally in combination with oat xylitol, in cosmetic compositions for increasing the ratio of commensal bacteria to harmful or pathogenic bacteria in human skin microbiome. Typically, the combined amount of meadowsweet extract and roseroot extract and/or dried forms thereof in a cosmetic composition is 0.02-20%, preferably 0.2-20% by weight of the cosmetic composition.

### Birch Sap

Birch sap (birch juice) is the sap directly tapped from birch trees (Betula spp) in early spring. Traditionally, it has been used in the Northern countries as a beverage. Use in traditional medicine and in cosmetics is also documented.

In one or more embodiments, the cosmetic composition for use in the present invention comprises birch sap or its dried form, preferably in an amount of 0.01-30% by weight of the composition, more preferably in an amount of 0.1-20% by weight of the composition, and a cosmetically acceptable carrier.

The birch sap used in the present invention is typically obtained by a method comprising the steps of collecting birch sap from Nordic birches in early spring, filtering the collected birch sap, and optionally drying the obtained birch sap, typically by spray-drying, to obtain birch sap powder.

In one or more embodiments, the invention relates to the non-therapeutic use of birch sap, optionally in combination with oat xylitol, in a cosmetic composition for increasing the ratio of commensal bacteria to harmful or pathogenic bacteria in human skin microbiome. Preferably, the amount of birch sap in the cosmetic composition is 1-20% by weight of the composition. If present, the amount of oat xylitol in the cosmetic composition comprising birch sap is preferably 0.1-5% by weight of the composition, typically at least 0.5%, such as 0.5-5%, by weight of the composition.

In some embodiments, the cosmetic composition for use in the present invention may comprise birch sap, meadowsweet extract and roseroot extract or dried forms thereof, optionally in combination with oat xylitol.

Chaga (Inonotus obliquus)

Chaga is a fungus growing on some deciduous trees, typically on birch. In the Nordic countries, it is either found growing wild on birch (Betula spp) trees or cultivated by inoculating growing birch trees with a chaga inoculum.

Typically, the chaga extract used in the present invention has been obtained by a method comprising the steps of crushing chaga obtained from Nordic birch trees, extracting the crushed chaga at room temperature with a solvent, preferably with water: ethanol, with circulating glycerine: water, with water: butylene- or pentyleneglycol, propanediol, to obtain a primary extract, which is filtrated and concentrated to obtain a concentrated chaga extract with a chaga content of 10-60%, typically about 40% by weight, and optionally drying the obtained chaga extract, for example by spray-drying.

In one or more embodiments, the cosmetic compositions used in the method of invention comprise concentrated (40%) chaga extract or its dried form, preferably in an amount of 0.01-10% by weight of the composition, more preferably in an amount of 0.1-5% by weight of the composition, and a cosmetically acceptable carrier.

### Fermented oat

Oat (Avena sativa) is a species of cereal grain primarily grown for its seed (oats). Oats are mainly used for human consumption and as livestock feed. However, recently the cosmetic use of oats, for example in the form of various oat extracts, oat xylitol and oat oil has increased.

In the present invention it was found that fermented oat increases the growth of commensal bacteria and may reduce the growth of harmful or pathogenic bacteria of human skin microbiome. The effect may be enhanced by the action of other Nordic plant-based ingredients, such as oat xylitol and birch sap.

In one or more embodiments, the cosmetic composition for use in the present invention comprises fermented oat extract or its dried form, preferably in an amount of 0.01-10% by weight of the composition, more preferably in an amount of 0.1-5% by weight of the composition, and a cosmetically acceptable carrier.

Typically, the fermented oat extract used in the present invention has been obtained by a method comprising the steps of fermenting Avena sativa (oat) kernels by lactic acid bacteria, separating any precipitates and optionally filtering the remaining solution to obtain the fermented oat extract. Optionally, the fermented oat extract may be dried, for example by spray-drying, to obtain fermented oat extract powder.

Oat xylitol

Recently, oat hulls, which are derived as a side stream from oat milling process, have been used in the production of another plant-based Nordic raw material, oat xylitol. In brief, oat hulls are hydrolysed, the obtained hydrolysate is separated to a solid fraction and to a carbohydrate containing liquid fraction, which is rich in xylose and may be used in the production of xylitol.

Previously, xylitol has been used in cosmetic products mainly as a moisturizing agent. In the present invention, xylitol derived from Nordic oat hulls is typically used in the cosmetic compositions in combination with at least one of the above-mentioned Nordic plant-based ingredients for increasing the ratio of commensal bacteria to harmful or pathogenic bacteria in human skin microbiome. For example, the following combinations have been found to be effective in increasing the ratio of commensal bacteria to harmful or pathogenic bacteria in human skin microbiome: xylitol and birch sap, xylitol and meadowsweet, xylitol and chaga, in particular xylitol and meadowsweet. It was also found that oat xylitol alone inhibits the growth of C. acnes.

The amount of xylitol in the cosmetic compositions for use in the methods of the invention is 0.01-50% by weight of the composition, preferably 0.1-5% by weight of the composition.

The non-therapeutic method and the non-therapeutic use of the invention may involve various topical cosmetic compositions comprising an effective amount substance(s) derived from Nordic plants. Examples of suitable topical cosmetic compositions include but are not limited to day creams, night creams, eye creams, moisturizers, face fresheners, serums, ampoules, toners, skin lotions, solid products like sticks, cloth faces, mask products, cleanser formulations, facial makeup and color cosmetics, hair products (wash and care products, styling products, masks), deodorants, disinfectants or the like.

The cosmetic compositions may also include any one of or any combination of the following additional cosmetically acceptable ingredients: water, moisturizing agents, preservatives, thickening agents, structuring agents, oils, vitamins, antioxidants, emulsifiers, waxes, fragrances, solubilizants and other cosmetically acceptable ingredients as known by persons skilled in the art and as appropriate for each type of various cosmetic compositions.

As stated above, the invention also relates to a cosmetic composition for use in a non-therapeutic method for increasing the ratio of commensal bacteria to harmful bacteria in human skin microbiome, wherein the cosmetic composition comprises an effective amount of at least one substance derived from Nordic plants, wherein said substance(s) comprise extracts or dried extracts of meadowsweet, and roseroot, optionally together with chaga, fermented oat or its dried form, birch sap or its dried form, or any combinations thereof, and further optionally in combination with oat xylitol.

In embodiments, the cosmetic compositions for use in the method of the invention may comprise in particular the following combinations of substances derived from Nordic plants:
roseroot and meadowsweet
birch sap and oat xylitol
birch sap and oat xylitol and fermented oat
roseroot and chaga, optionally with oat xylitol
meadowsweet and chaga, optionally with oat xylitol
birch sap and roseroot, optionally with oat xylitol
birch sap and meadowsweet, optionally with oat xylitol
birch sap and chaga, optionally with oat xylitol

In further embodiments, the following combinations are preferred:
roseroot and meadowsweet
birch sap and oat xylitol
birch sap and oat xylitol and fermented oat extract
roseroot and chaga, optionally with oat xylitol
meadowsweet and chaga, optionally with oat xylitol

In one preferred embodiment, the cosmetic combination comprises roseroot and meadowsweet, preferably in the form of liquid extracts thereof.

Depending on the particular substance, the above combinations of substances derived from Nordic plants include the substances for example in the form of (liquid) extracts or dried extracts (powders). Typically, roseroot, meadowsweet and chaga are provided in the form of liquid extracts thereof. Oat xylitol is preferably provided in powder form. Birch sap and fermented oat extract are preferably included in the cosmetic compositions in liquid form. However, also dried forms of the above-mentioned liquid forms may be used, where applicable or desired.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

### EXPERIMENTAL

### Example 1. Laboratory Tests

In the present study, the effects of 4 compounds and 5 combinations were evaluated on the growth of two bacterial strains: Staphylococcus epidermidis (S. epidermidis) and Cutibacterium acnes (C. acnes). The bacterial strains were grown individually, and the growth of each bacterial strain was determined in presence of the compounds or combinations by optical density (OD) measurement at 600 nm in kinetic for 24 hours for S. epidermidis strain and for 49 hours for C. acnes strain to determine a bacterial growth curve.

Tested compounds and combinations are listed in Table 1 and Table 2, respectively.

TABLE 1
Test compounds and their concentrations

| Test compound | Type/storage | Test concentration |
|---|---|---|
| Xylitol | Powder, storage RT | Tested alone at 1 and 5% |
| | | and in combination at 0.5% |
| | | Only tested in combination |
| | | at 0.5% |
| Meadowsweet | Liquid, storage +4° C. | Tested alone at |
| extract | | 0.2, 0.5, 1, 2, |
| | | and 5% and |
| | | in combination |
| Birch sap | Liquid, storage −20° C. | Tested alone at |
| | | 1, 5, 10, 15, |
| | | and 20% and |
| | | in combination |
| Fermented | Homogenous | Only tested in combination |
| oat extract* | turbid liquid, | at 0.5% |
| | storage +4° C. | |
| Roseroot extract | Liquid, storage +4° C. | Tested alone at |
| | | 0.2, 0.5, 1, 2, |
| | | and 5% and |
| | | in combination |
| Chaga extract** | Viscous liquid, | Tested alone at |
| | storage +4° C. | 0.25, 0.5, 1, |
| | | 2, and 5% and in |
| | | combination |

*Fermented oat extract (dissolved in assay medium) was filtered to remove Lactobacillus from the liquid.
**Chaga extract (dissolved in assay medium) was filtered to remove microorganisms from the liquid.

TABLE 2
Tested combinations and their concentrations

| Combination | Test concentrations |
|---|---|
| Xylitol + Birch sap | 0.5% + 1%, 0.5% + 5%, 0.5% + 10%, |
| | 0.5% + 15%, 0.5% + 20% |
| Xylitol + Birch sap + | 0.5% + 1% + 0.5%, 0.5% + 5% + 0.5%, |
| Fermented oat extract | 0.5% + 10% + 0.5%, 0.5% + 15% + 0.5%, |
| | 0.5% + 20% + 0.5% |
| Xylitol + | 0.5% + 0.2%, 0.5% + 0.5%, 0.5% + 1%, |
| Meadowsweet extract | 0.5% + 2%, 0.5% + 5% |
| Xylitol + | 0.5% + 0.2%, 0.5% + 0.5%, 0.5% + 1%, |
| Roseroot extract | 0.5% + 2%, 0.5% + 5% |
| Xylitol + | 0.5% + 0.25%, 0.5% + 0.5%, 0.5% + 1%, |
| Chaga extract | 0.5% + 2%, 0.5% + 5% |

To determine the effect of the test compounds and combinations on the growth of bacteria, bacterial suspensions were transferred in a 96-well plate containing the compounds, the combinations or glucose tested at 0.1% and 1%. Bacterial cultures were incubated at 37° C. under agitation and under anaerobic condition for C. acnes strain. All experimental conditions were performed in n=3.

Optical densities at 600 nm (OD600 nm) were then read in kinetic for 24 hours for S. epidermidis strain and 49 hours for C. acnes strain using a microplate spectrophotometer (EPOCH2, BioTek Instruments). Raw data were analysed using GraphPad PRISM software (growth curves).

### Results

As explained above, the growth-promoting or growth-inhibiting effects of these extracts were investigated at different concentrations in two different bacterial species, Staphylococcus epidermidis and Propionibacterium acnes, now known as Cutibacterium acnes. S. epidermidis is part of the normal flora of human skin, which is not pathogenic on healthy skin but a commensal bacterium. Cutibacterium acnes is a normally harmless bacterium on the skin, but known to be associated with acne. This bacterium thrives on adolescent skin because of its increased fat content, which favours this bacterium.

Overall, under the experimental conditions of this assay and depending on the concentration, all tested compounds tended to stimulate the growth of S. epidermidis whereas they tended to inhibit the growth of C. acnes. Detailed results are discussed below.

### Birch Sap

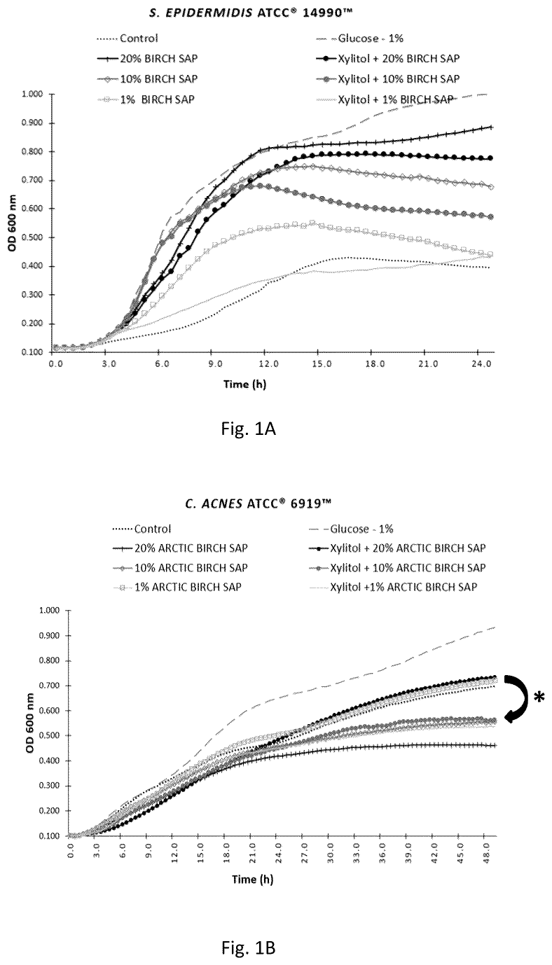

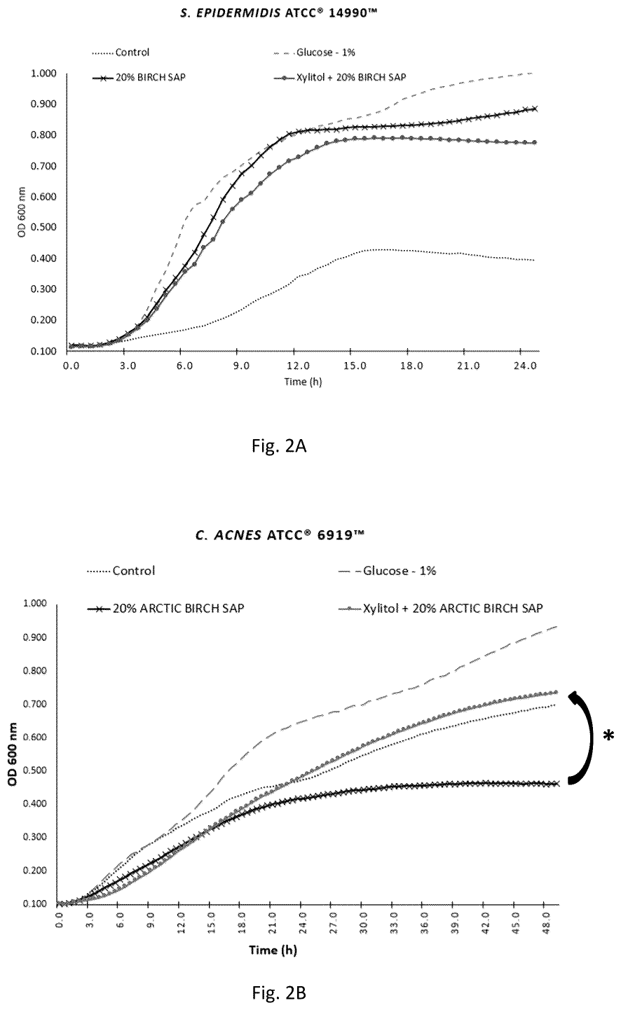

Effect of birch sap alone and in combination with oat xylitol on the growth of S. epidermidis and C. acnes is shown in FIGS. 1 and 2.

As can be seen in FIGS. 1A and 1B, growth of S. epidermidis is induced by arctic birch sap (20-1%) alone and with xylitol when birch sap concentration is over 1%. The growth of C. acnes is reduced by arctic birch sap (20% and 10%). Xylitol potentiates 10-fold the growth inhibition of 1% birch sap against C. acnes as the smallest concentration of birch sap (1%) with xylitol produces equal growth inhibition as 10% birch sap alone (arrow* right panel of FIG. 1B). The lowest concentration 1% of birch sap+xylitol does not increase S. epidermidis growth, but maintains control growth rate.

The effect of birch sap at 20% concentration on the growth of S. epidermidis and C. acnes in vitro, alone and in combination with xylitol, is shown in FIGS. 2A and 2B. The growth of S. epidermidis is induced by 20% arctic birch sap and xylitol, whereas C. acnes growth is reduced by arctic birch sap (20%). Thus at the highest concentration of birch sap (20%) xylitol hinders the growth inhibition of birch sap against C. acnes as the growth returns to control level (arrow* right panel of FIG. 2B).

As a summary, arctic birch sap acts as a prebiotic in suitable concentrations, increasing the growth of S. epidermidis in vitro. Interestingly, arctic birch sap, in turn, inhibits the growth of C. acnes. Xylitol can potentiate 10-fold the growth inhibitory effect of birch sap at the lowest concentration. In the highest concentration of 20% birch sap alone is more effective inhibitor of C. acnes growth than with xylitol.

### Fermented Oat Extract

A commercial product, lactobacilli fermented oat kernel extract (aurafirm N), was used in the study. According to the manufacturer's INCI list, the product contains aqua, Lactobacillus ferment, Avena sativa (oat) kernel extract, sodium benzoate and potassium sorbate.

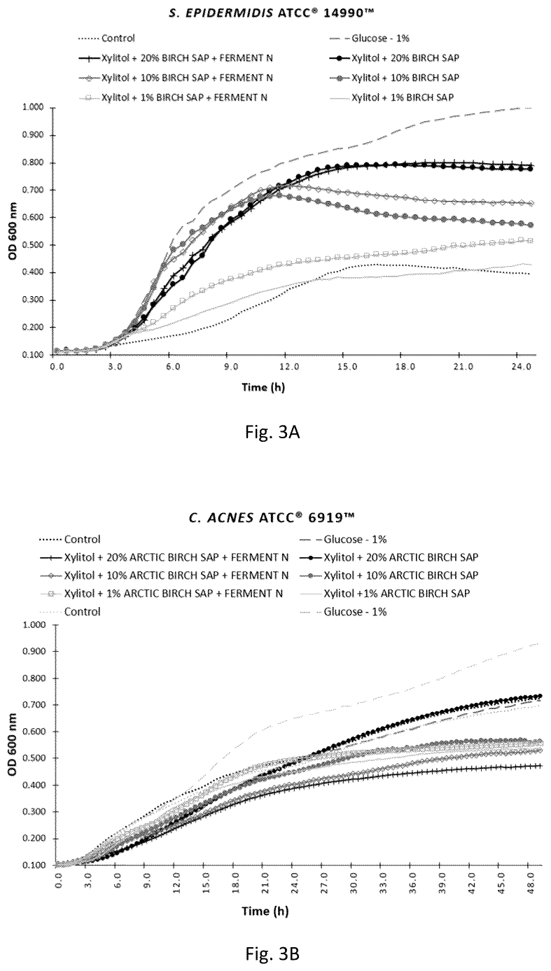

Effect of fermented oat extract (“Ferment N”) in combination with oat xylitol and birch sap on the growth of S. epidermidis and C. acnes is shown in FIGS. 3A and 3B.

Based on the results illustrated in FIGS. 3A and 3B, fermented oat extract may increase slightly the growth of S. epidermidis at smaller concentrations of birch sap and xylitol. The difference seen in C. acnes 20% birch sap+xylitol+/− fermented oat extract may be caused by differences in the two 96-well plates.

### Arctic Meadowsweet

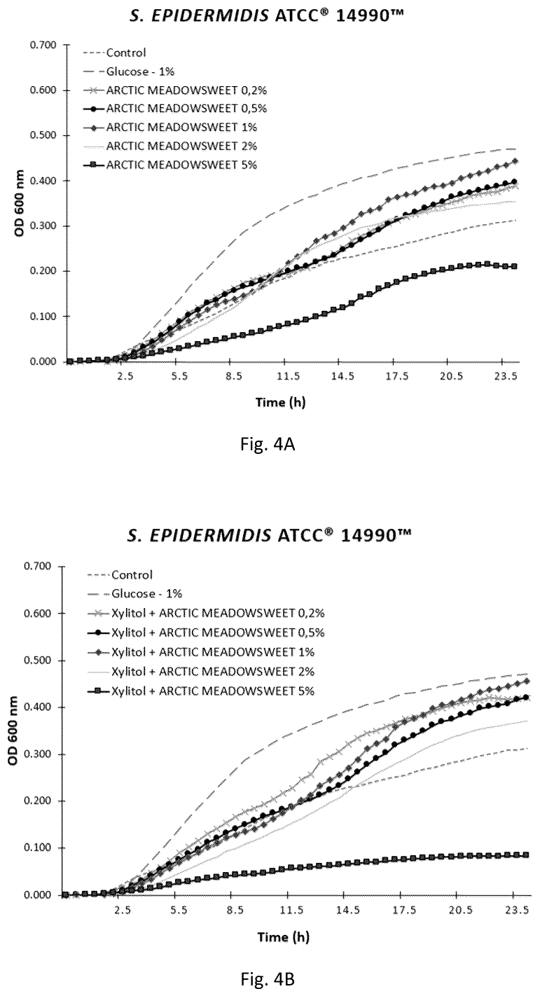

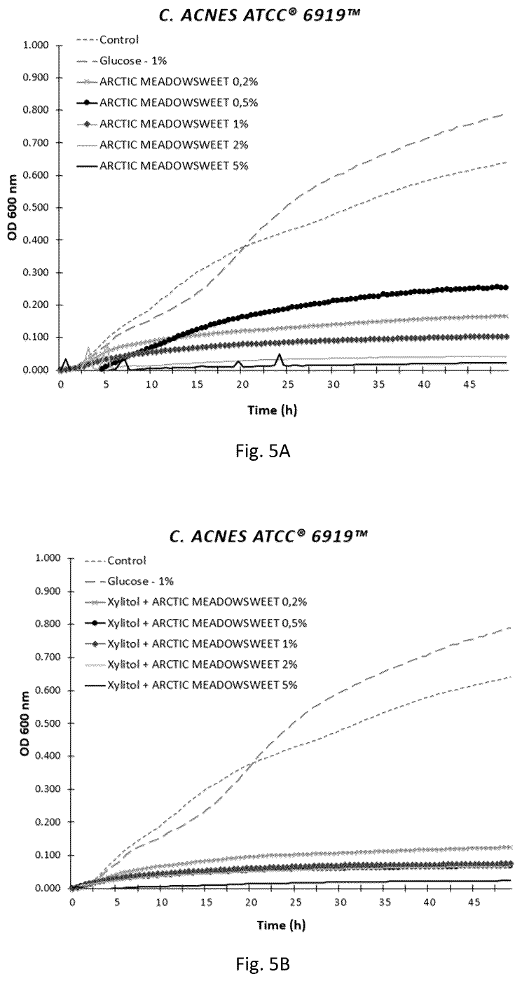

Effects of arctic meadowsweet alone and in combination with oat xylitol on the growth of S. epidermidis and C. acnes in laboratory conditions are shown in FIGS. 4 and 5.

According to the results presented in FIG. 4A, the growth of S. epidermidis was slightly increased at low concentrations of arctic meadowsweet. The highest concentration of arctic meadowsweet (5%) with xylitol inhibited S. epidermidis growth (FIG. 4B). On the other hand, as shown in FIG. 5A, the growth of C. acnes is clearly reduced at all different concentrations of the arctic meadowsweet. Xylitol further slightly potentiates the growth-reducing effect on C. acnes with arctic meadowsweet at all concentrations (FIG. 5B).

Arctic roseroot

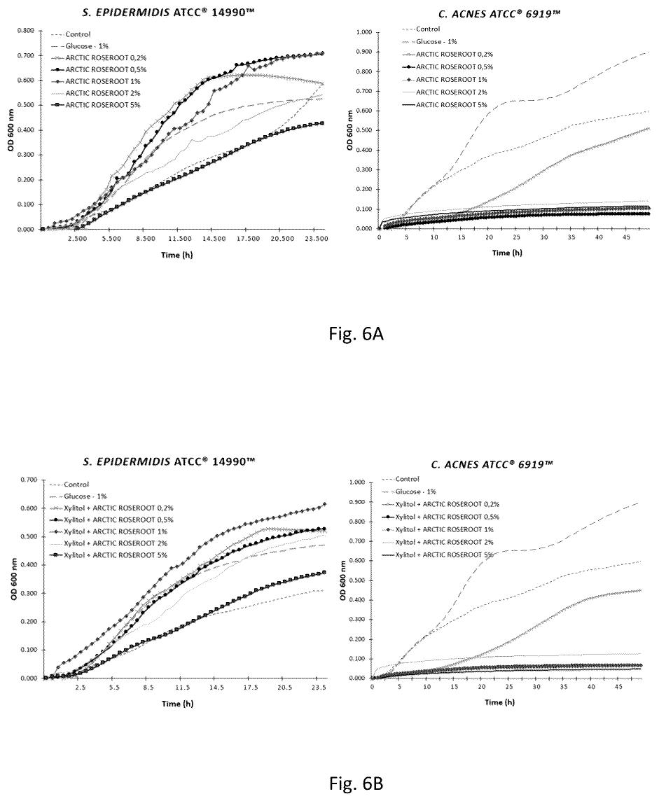

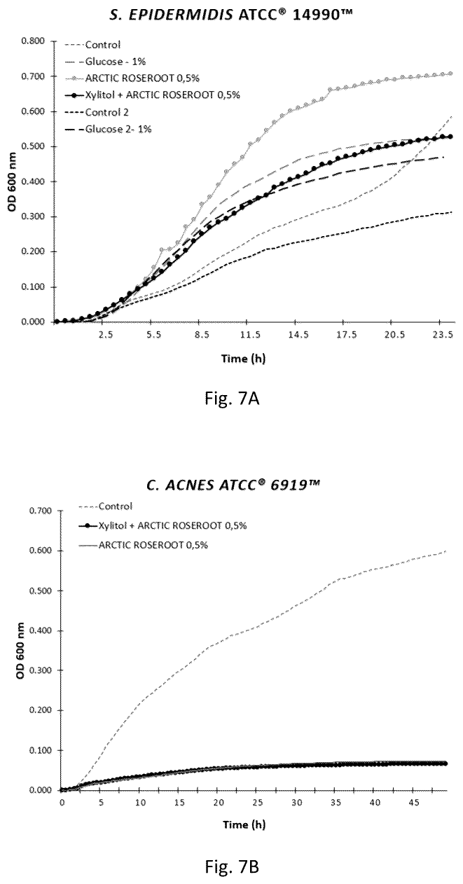

FIG. 6 illustrates the effect of arctic roseroot alone (FIG. 6A) and in combination with oat xylitol (FIG. 6B) on the growth of S. epidermidis and C. acnes in laboratory conditions. FIG. 7 shows the effect when the concentration of arctic roseroot is 0.5%.

Arctic roseroot extract was found to have a growth-enhancing effect on S. epidermidis and a growth-reducing effect on the growth of C. acnes bacteria. This is favourable tendency for oily and acne prone skin. Oat xylitol does not seem to provide any additive effect at least for C. acnes.

### Chaga

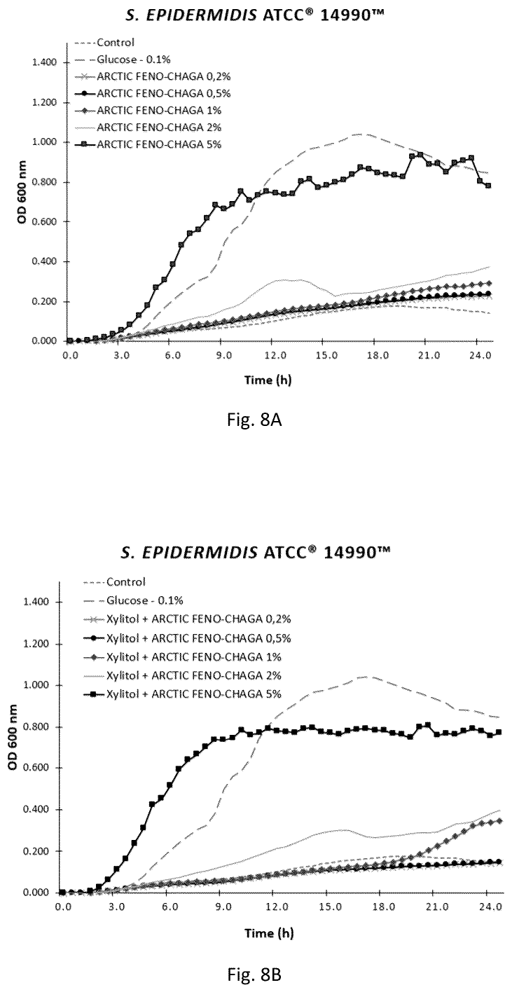

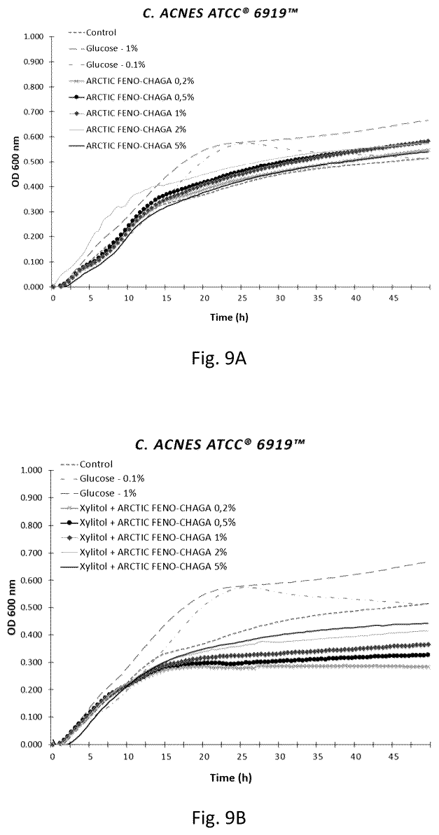

Effect of arctic chaga alone and in combination with oat xylitol on the growth of S. epidermidis and C. acnes in laboratory conditions is s shown in FIGS. 8 and 9.

The highest concentration of chaga increases S. epidermidis growth highly (FIG. 8A). Xylitol did not potentiate the effect of chaga (FIG. 8B).

FIG. 9B shows that xylitol and chaga (0,2 and 0.5%) together drive the C. acnes growth earlier to the stationary phase. The combination 0.5% chaga and 0.5% xylitol decrease C. acnes growth and maintain S. epidermidis growth at control level.

To summarize, several interesting effects of the above disclosed northern raw materials on the growth of microbes living on the skin were found in the laboratory tests. The most promising findings concerned the use of organic birch sap, arctic meadowsweet, roseroot and chaga. Extracts from said plants were found to have a prebiotic effect, increasing the growth of a favourable commensal, S. epidermidis, under laboratory conditions, while providing a growth-reducing effect on the bacterium C. acnes. Oat xylitol may further potentiate said effects. These results open many applications for these northern plant extracts in cosmetics and especially in the cosmetic treatment of oily and combination skin.

### Example 2. In Vivo Clinical Study

Next, one cosmetic skin serum product comprising arctic meadowsweet and roseroot was tested in cosmetic treatment of impure oily and combination skin. The in vitro growth assays had shown a clear increase in S. epidermidis growth with roseroot and a neutral effect with meadowsweet. Both of the ingredients reduced C. acnes growth in vitro.

A cosmetic skin care serum was formulated to contain 0.5% arctic meadowsweet and 0.5% arctic roseroot extracts. Other ingredients of the serum included typical ingredients of skin care serums, such as aqua, humectants, thickeners, preservatives, colorants and perfume.

The product was used twice a day, in the morning and in the evening, by healthy volunteers in three separate studies (n=19, n=25, n=27) as a facial care for 28 days. In the beginning and at the end of the study swaps were taken from cheeks of the subjects for quantification of total flora, S. epidermidis and C. acnes by qPCR. In addition, a subjective evaluation was carried out by using a questionnaire.

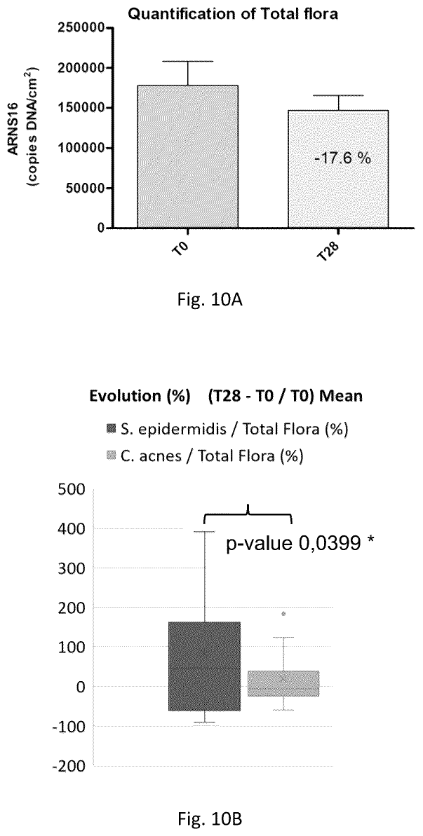

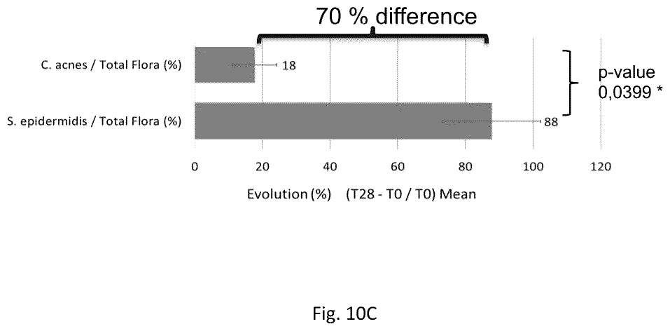

The results from quantification of total flora, S. epidermidis and C. acnes with qPCR are shown in FIG. 10. The relative number of S. epidermidis as compared to total flora was increased and the relative number of C. acnes was reduced during the one month in vivo use when quantified by qPCR (n=27) from swap samples taken from the cheeks of subjects that had been using the product for 28 days, twice per day.

In the in vivo clinical study the number of clogged pores (comedoes), papules and pustulas (retentional and inflammatory lesions) were counted before and after 1 month use of the product. Also, skin redness and gloss were evaluated and measured by experts. It was found that after approximately one month's use the product comprising arctic roseroot and meadowsweet provided several effects which make it suitable for impure, oily or combination skin:
the look of pores and redness was diminished
skin mattifying effect
reduction in global retentional and inflammatory lesions
non-comedogenic effect

To conclude, the in vivo clinical trial results supported the in vitro results by showing a reduction in the relative quantity of C. acnes and increase of S. epidermidis in vivo as analysed by qPCR from swaps taken from the cheeks after 28 day use of a serum formulated to contain arctic meadowsweet and roseroot. Also, other in vivo measurements from clinical trials supported the favourable profile of arctic meadowsweet and roseroot for oily and combination, impure skin; that is, the product diminished the look of pores and redness, gave a mattifying effect and reduced the number of global retentional and inflammatory lesions. Based on the clinical study, the product can be considered as non-comedogenic and non-acnegenic. These are important features for a cosmetic product designed for combination and oily skin type, which easily develops clogged pores (comedoes), papules and inflamed pustules (retentional impurities). All these clinical in vivo results support the finding that arctic meadowsweet and roseroot are selective prebiotics for the favourable commensal, S. epidermidis, and inhibitory for the potential pathogen C. acnes.

Thus, both the in vitro and in vivo data give logical results that show the usefulness of arctic roseroot and meadowsweet in cosmetic skincare products, in particular in those meant for impure, oily or combination skin.

### Formulation Examples

Example 3: Skin care cream, lotion or gel

| INGREDIENTS | Max. levels % (w/w) |
|---|---|
| Oils, waxes and fats | 30 |
| Humectants | 20 |
| Thickeners | 12 |
| Cosmetic ingredients from | 0.01-20 |
| Nordic plants as defined above | |
| Bulking agents | 5 |
| UV filters | 5 |
| Emulsifying agents | 5 |
| Preservatives | 2 |
| Colorants | 2 |
| Perfume | 1 |
| Aqua | to 100 |

Example 4. Zinc oxide based cream

| INGREDIENTS | Max. levels % (w/w) |
|---|---|
| Oils, waxes and fats | 60 |
| Bulking agents | 30 |
| Zinc oxide | 25 |
| Humectants | 20 |
| Emulsifying agents | 12 |
| Cosmetic ingredients from | 0.01-20 |
| Nordic plants as defined above | |
| Thickeners | 5 |
| Preservatives | 1.5 |
| Perfume | 1 |
| Aqua | to 100 |

Example 5. Tonic cosmetic water

| INGREDIENTS | Max. levels % (w/w) |
|---|---|
| Humectants | 25 |
| Cosmetic ingredients from | 0.01-20 |
| Nordic plants as defined above | |
| Oils (e.g. vegetable oils) | 50 |
| Additional ingredients | 5 |
| Emulsifying agents | 5 |
| Thickeners | 2 |
| Preservatives | 2 |
| Perfume | 1.5 |
| Colorants | 1 |
| Aqua | to 100 |

Example 6. Lip care product or lip colour
product (stick, cream or gel)

| INGREDIENTS | Max. levels % (w/w) |
|---|---|
| Oils, waxes and fats | 90 |
| Humectants | 50 |
| Aqua | 50 |
| Film forming agents | 30 |
| Thickeners | 15 |
| UV filters | 15 |
| Bulking agents (e.g. silica) | 10 |
| Emulsifying agents | 5 |
| Cosmetic colorants | 5 |
| Cosmetic ingredients from | 0.01-20 |
| Nordic plants as defined | |
| above | |
| Perfume | 2 |
| Preservatives, antimicrobials | 1 |
| Flavouring agents | 1 |

Example 7. Cleansing gel

| INGREDIENTS | Max. levels % (w/w) |
|---|---|
| Anionic surfactants | 20 |
| Oils, waxes and fats | 20 |
| Humectants | 20 |
| Non-ionic/amphoteric surfactants | 15 |
| Foam boosting agents | 5 |
| Cosmetic ingredients from | 0.01-20 |
| Nordic plants as defined above | |
| Thickeners | 5 |
| Preservatives | 2 |
| Perfume | 1 |
| Colorants | 1 |
| Aqua | to 100 |

Example 8. Foundation

| INGREDIENTS | Max. levels % (w/w) |
|---|---|
| Isoparaffin and volatile emollients, | 60 |
| like C15-19 alkanes and C13- | |
| alkanes | |
| Oils | 40 |
| Emollients (e.g. plant esters) | 30 |
| Silicones | 30 |
| Bulking agents | 30 |
| Colorants, color additives | 30 |
| Humectants | 25 |
| Thickeners, emulsifying agents | 20 |
| UV filters | 10 |
| Surfactants | 5 |
| Cosmetic ingredients from | 0.01-20 |
| Nordic plants as defined above | |
| Perfume | 1 |
| Preservatives | 1 |
| Aqua | to 100 |

Example 9. Mascaras

| INGREDIENTS | Max. level % (w/w) |
|---|---|
| Silicones excl. volatile silicones | 80 |
| Oils, waxes and fats | 30 |
| Resins, film forming polymers | 30 |
| Colorants, colour additives | 25 |
| Emulsifying agents (e.g. stearates) | 20 |
| Bulking agents | 15 |
| Thickeners | 12 |
| Aqua | 60 |
| Humectants | 5 |
| Cosmetic ingredients from | 0.01-20 |
| Nordic plants as defined above | |
| Perfume | 1 |
| Antioxidants | 1 |
| Preservatives | 1 |

Example 10. Skin care serum

| INGREDIENTS | Max. levels % (w/w) |
|---|---|
| Humectants | 30 |
| Thickeners | 5 |
| Cosmetic ingredients from | 0.01-20 |
| Nordic plants as defined above | |
| Preservatives, antimicrobials | 2 |
| Colorants | 2 |
| Perfume | 1 |
| Aqua | to 100 |

As will be understood from the preceding description of the present invention and the illustrative experimental examples, the present invention can be described by reference to the following embodiments:
1. Non-therapeutic use of substances derived from Nordic plants in cosmetic compositions for increasing the ratio of commensal bacteria to harmful or pathogenic bacteria in human skin microbiome, wherein the substances derived from Nordic plants are selected from extracts and dried extracts of meadowsweet, roseroot and chaga, fermented oat and its dried form, birch sap and its dried form, and any combinations thereof, optionally in combination with oat xylitol.
2. The non-therapeutic use according to embodiment 1 for increasing the ratio of Staphylococcus epidermidis to harmful or pathogenic bacteria in human skin microbiome, in particular for increasing the ratio of Staphylococcus epidermidis to Cutibacterium acnes (Propionibacterium acnes).
3. The non-therapeutic use according to embodiment 1 or 2, wherein the cosmetic composition comprises meadowsweet extract or dried meadowsweet extract, preferably in an amount of 0.01-10% by weight of the composition, more preferably in an amount of 0.1-5% by weight of the composition, and a cosmetically acceptable carrier.
4. The non-therapeutic use according to any one of the preceding embodiments, wherein the meadowsweet extract has been obtained from flowers of meadowsweet by a method comprising the steps of extracting the flowers with a solvent, preferably with circulating glycerin: water, with water: butylene, with pentyleneglycol, propanediol or ethanol, more preferably with circulating glycerine: water at room temperature, and optionally drying the obtained meadowsweet extract, typically by spray-drying.
5. The non-therapeutic use according to any one of the preceding embodiments, wherein the cosmetic composition comprises roseroot extract or dried roseroot extract, preferably in an amount of 0.01-10% by weight of the composition, more preferably in an amount of 0.1-5% by weight of the composition, and a cosmetically acceptable carrier.
6. The non-therapeutic use according to any one of the preceding embodiments, wherein the roseroot extract has been obtained from roots of roseroot plants by a method comprising the steps of extracting the roots with a solvent, preferably with circulating glycerine: water, with water: butylene, with pentyleneglycol, propanediol or ethanol, more preferably with circulating glyecerine: water at room temperature, and optionally drying the obtained roseroot extract, typically by spray-drying.
7. The non-therapeutic use according to any one of the preceding embodiments, wherein the cosmetic composition comprises meadowsweet extract and roseroot extract and/or dried forms thereof.
8. The non-therapeutic use according to any one of the preceding embodiments, wherein the cosmetic composition comprises birch sap or its dried form, preferably in an amount of 0.01-30% by weight of the composition, more preferably in an amount of 0.1-20% by weight of the composition, and a cosmetically acceptable carrier.
9. The non-therapeutic use according to any one of the preceding embodiments, wherein the birch sap has been obtained by a method comprising the steps of collecting birch sap from Nordic birches in early spring, filtering the collected birch sap, and optionally drying the obtained birch sap, typically by spray-drying.
10. The non-therapeutic use according to any one of the preceding embodiments, wherein the cosmetic composition comprises chaga extract or its dried form, preferably in an amount of 0.01-10% by weight of the composition, more preferably in an amount of 0.1-5% by weight of the composition, and a cosmetically acceptable carrier.
11. The non-therapeutic use according to any one of the preceding embodiments, wherein the chaga extract has been obtained by a method comprising the steps of crushing chaga obtained from Nordic birch trees, extracting the crushed chaga at room temperature with a solvent, preferably with water: ethanol, with circulating glycerine: water, with water: butylene- or pentyleneglycol, or propanediol, to obtain a primary extract, which is filtrated and concentrated to obtain a concentrated chaga extract, and optionally drying the obtained chaga extract, for example by spray-drying.
12. The non-therapeutic use according to any one of the preceding embodiments, wherein the cosmetic composition comprises fermented oat extract or its dried form, preferably in an amount of 0.01-10% by weight of the composition, more preferably in an amount of 0.1-5% by weight of the composition, and a cosmetically acceptable carrier.
13. The non-therapeutic use according to any one of the preceding embodiments, wherein the fermented oat extract has been obtained by a method comprising the steps of fermenting Avena sativa (oat) kernels by lactic acid bacteria, separating any precipitates and optionally filtering the remaining solution to obtain the fermented oat extract.
14. The non-therapeutic use according to any one of the preceding embodiments, wherein the substance(s) derived from Nordic plants is/are used in the cosmetic compositions in combination with oat xylitol obtained by processing Nordic oat hulls.
15. The non-therapeutic use according to embodiment 14, wherein the amount of oat xylitol in the cosmetic compositions is 0.01-50% by weight of the composition, preferably 0.1-5% by weight of the composition.
16. The non-therapeutic use according to any one of the preceding embodiments for increasing the ratio of S. epidermidis to (′. acnes, typically from 1:1 to at least 5:1 when the cosmetic composition is applied on the human skin for one month.
17. A non-therapeutic cosmetic method for increasing the ratio of commensal bacteria to harmful or pathogenic bacteria in human skin microbiome, the method comprising applying on the skin a topical cosmetic composition comprising an effective amount of substance(s) derived from Nordic plants, wherein said substances are selected from extracts and dried extracts of meadowsweet, roseroot and chaga, fermented oat and its dried form, birch sap and its dried form, and any combinations thereof, optionally in combination with oat xylitol.
18. The non-therapeutic method according to embodiment 17, wherein the effective amount of substance(s) derived from Nordic plants is 0.01-70% by weight of the composition, preferably 0.1-20% by weight of the composition.
19. A cosmetic composition for use in a non-therapeutic method for increasing the ratio of commensal bacteria to harmful bacteria in human skin microbiome, wherein the cosmetic composition comprises an effective amount of at least one substance derived from Nordic plants, wherein said substance(s) is/are selected from extracts and dried extracts of meadowsweet, roseroot and chaga, fermented oat and its dried form, birch sap and its dried form, and any combinations thereof, optionally in combination with oat xylitol.
20. The cosmetic composition for use according to embodiment 19, wherein the cosmetic composition comprises meadowsweet extract and roseroot extract, together with other cosmetically acceptable substances.
21. The cosmetic composition for use according to embodiment 20, wherein the combined amount of meadowsweet extract and roseroot extract in the cosmetic composition is 0.02-20%, preferably 0.2-20% by weight of the cosmetic composition

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in cosmetic industry.

### Acronyms List

OD optical density
qPCR quantitative polymerase chain reaction

### CITATION LIST

### Patent Literature

EP 1337224
EP 2814242
EP 3365068
FR 2897778 A1
US 2020/004663 A1

### Non Patent Literature

Byrd A, Belkaid Y, Segre J. (2018) The human skin microbiome. Nat Rev Microbiol 16, 143-155
Callewaert C, Kerckhof F-M, Granitsiotis M S, Van Gele M, Van de Wiele T, et al. (2013) Characterization of Staphylococcus and Corynebacterium Clusters in the Human Axillary Region. PLOS ONE 8 (8)
Davis C P. (1996) Chapter 6. Normal Flora. In Baron S (ed.) Medical Microbiology, 4th ed. Galveston. Texas
Iwase T, Uehara Y, Shinji H, Tajima A, Seo H, Takada K, Agata T, Mizunoe Y. (2010). Staphylococcus epidermidis Esp inhibits Staphylococcus aureus biofilm formation and nasal colonization. Nature 465, 346-349
Wang Y, Kuo S, Shu M, Yu J, Huang S, Dai A, Two A, Gallo R L, Huang C M. (2014). Staphylococcus epidermidis in the human skin microbiome mediates fermentation to inhibit the growth of Propionibacterium acnes: Implications of probiotics in acne vulgaris. Appl Microbiol Biotechnol. 98 (1): 411-424

1-24. (canceled)

25. A method for treating human skin, the method comprising:
applying a composition to the skin of a human, the composition comprising substances derived from Nordic plants,
wherein the substances derived from Nordic plants comprise meadowsweet and roseroot, optionally in combination with oat xylitol, and
wherein the composition is effective in increasing the ratio of commensal bacteria to harmful or pathogenic bacteria in the human skin microbiome.

26. The method of claim 25, wherein the composition is effective in increasing the ratio of Staphylococcus epidermidis to harmful or pathogenic bacteria in the human skin microbiome, and wherein the harmful or pathogenic bacteria in the human skin microbiome comprise Cutibacterium acnes (Propionibacterium acnes).

27. The method according to claim 26, wherein the composition is effective to increase the ratio of Staphylococcus epidermidis to Cutibacterium acnes from 1:1 to at least 5:1.

28. The method according to claim 25, wherein the meadowsweet is provided in an amount of 0.01-10% by weight of the cosmetic composition.

29. The method according to claim 25, wherein the meadowsweet and/or roseroot are provided in the form of an extract or a dried extract of the meadowsweet and/or roseroot.

30. The method according to claim 29, wherein the roseroot is provided in an amount of 0.01-10% by weight of the cosmetic composition.

31. The method according to claim 31, wherein the substances derived from Nordic plants further comprise birch sap or its dried form.

32. The method according to claim 31, wherein the birch sap is obtained by collecting birch sap from Nordic birches, filtering the collected birch sap, and optionally drying the obtained birch sap.

33. The method according to claim 25, wherein the substances derived from Nordic plants further comprise chaga extract or its dried form.

34. The method according to claim 33, wherein the chaga extract is obtained by crushing chaga obtained from Nordic birch trees, extracting the crushed chaga at room temperature with a solvent, to obtain a primary extract, which is filtrated and concentrated to obtain a concentrated chaga extract, and optionally drying the obtained chaga extract.

35. The method according to claim 25, wherein the substances derived from Nordic plants further comprise a fermented oat extract or its dried form.

36. The method according to claim 35, wherein the fermented oat extract is obtained by fermenting Avena sativa (oat) kernels by lactic acid bacteria, separating any precipitates, and optionally filtering the remaining solution to obtain the fermented oat extract.

37. The method according to claim 25, wherein the composition further comprises oat xylitol, obtained by processing Nordic oat hulls.

38. The method according to claim 37, wherein the amount of oat xylitol in the cosmetic compositions is 0.01-50% by weight of the composition.

39. The method according to claim 25, wherein the combined amount of meadowsweet extract and roseroot extract in the cosmetic composition is 0.02-20% by weight of the cosmetic composition.

40. The method according to claim 25, wherein the substances derived from Nordic plants further comprises chaga, fermented oat, and birch sap, and wherein the composition further comprises oat xylitol.

41. The method according to claim 25, wherein the amount of the one or more substances derived from Nordic plants is 0.01-70% by weight of the composition.

42. The method according to claim 41, wherein the amount of the one or more substances derived from Nordic plants is 0.1-20% by weight of the composition.

43. The method according to claim 25, wherein the composition further comprises a cosmetically acceptable carrier.

44. The method according to claim 25, wherein composition comprises:
a liquid extract of meadowsweet, wherein the amount of meadowsweet is from 0.01-10 weight % of the composition;
a liquid extract of roseroot, wherein the amount of roseroot is from 0.01-10 weight % of the composition;
a liquid extract of chaga, wherein the amount of chaga is from 0.01-10 weight % of the composition;
oat xylitol in powder form, wherein the amount of oat xylitol is from 0.01-50% by weight of the composition;
birch sap in liquid form, wherein the amount of birch sap is from 0.01-30% by weight of the composition; and
a fermented oat extract in liquid form, wherein the fermented oat extract comprises 0.01-10% by weight of the composition.
